(19) United States
(12) Patent Application Publication
(10) Pub. No.: US 2006/0216766 A1
(43) Pub. Date: Sep. 28, 2006

(54) Assay for protein isoforms

(75) Inventor: Philip Desmond Rye, Oslo (NO)

Correspondence Address: BACON & THOMAS, PLLC, 625 SLATERS LANE, FOURTH FLOOR, ALEXANDRIA, VA, 22314, US

(73) Assignee: Axis-Shield ASA, Oslo (NO)

(21) Appl. No.: 10/544,441

(22) PCT Filed: Feb. 6, 2004
(86) PCT No.: PCT/GB04/00480

§ 371 Date: Dec. 5, 2005

(30) Foreign Application Priority Data
Feb. 6, 2003 (GB) 0302740.6

(51) Int. Cl. G01N 33/53; C12Q 1/37

(52) U.S. Cl. 435/7.92; 435/23

(57) ABSTRACT

A method for assaying for a protein having at least two isoforms having different glycosylation patterns, the method including contacting a sample containing the protein with a proteolytic enzyme, and detecting the content or relative content of at least one peptide fragment produced by proteolysis of the protein.

This invention relates to an assay for proteins having two or more isoforms differing in their pattern of glycosylation, e.g. having glycosylated and non-glycosylated isoforms or fully and partially glycosylated isoforms, and to kits for such assays.

Various proteins exist in two or more different isoforms differing in their pattern of glycosylation. Such differences, or the relative proportions of the differently glycosylated isoforms, may be indicative of a disease or disorder or of substance abuse and thus there is a need for assay systems capable of distinguishing between the differently glycosylated isoforms.

The use of antibodies to distinguish between differently glycosylated isoforms of endogenous proteins is however relatively problematic as the success rate in raising antibodies which bind specifically or preferentially to particular isoforms of endogenous glycosylated proteins is relatively low.

One example where the determination of the relative concentrations of differently glycosylated isoforms of an endogenous protein is of clinical interest is the case of the blood protein transferrin. The amino acid backbone of transferrin contains two sites (Asn 413 and Asn 611) which may bear bi- or tri-antennary oligosaccharide side chains with terminal sialic acid groups. In a healthy patient, the majority of the blood transferrin molecules carry four or five sialic acid groups; however where the patient is an alcoholic the proportion of the transferrin molecules with no sialic acid groups or with two or three sialic groups is relatively increased. Indeed the absence of one or both complete glycan chains has also been shown to be a characteristic feature of transferrin isoforms in alcoholics. (See for example Arndt in Clinical Chemistry 47: 13-27 (2001)). Abnormal relative abundances of the transferrin isoforms also occur in patients with carbohydrate-deficient glycoprotein syndromes (CDGS) or congenital disorders of glycosylation (CDG), e.g. as discussed by Keir et al. in Ann. Clin. Biochem. 36: 20-36 (1999).

Various assays for such “carbohydrate-deficient transferrin” (CDT) or “carbohydrate-free transferrin” (CFT) have been proposed; however those suitable for automation generally rely on the use of an ion exchange resin to separate out the transferrin molecules with three or less sialic acid groups from those with four or five sialic acid groups on the basis of the different pHs at which the different isoforms are released from or taken up by the resin. Examples of such assays are described in U.S. Pat. No. 4,626,355 (Pharmacia), WO 96/26444 (Axis) and WO 01/42795 (Axis).

Any protein with post-translational glycosylation can occur in different glycosylation isoforms. Thus, besides transferrin other clinically relevant proteins exist in differently glycosylated isoforms, including glycosylated markers for cancers and other diseases, e.g. alkaline phosphatase (AP) (see Magnusson et al. Clinical Chemistry 44: 1621-1628 (1998)), alpha-fetoprotein (AFP), human chorionic gonadotropin (HCG), and possibly also prion protein (CD230).

Mammalian alkaline phosphatases comprise a ubiquitous family of enzymes. AP is a glycoprotein enzyme, residing in the outer leaflet of the cytoplasmic membrane where a glycosyl phosphatidylinositol moiety serves as a membrane anchor. The (native) molecular mass of liver AP, bone AP, and kidney AP has been determined as 152, 166 and 168 kDa respectively. Apart from its role in normal bone mineralization, other functions of L/B/K AP in physiological and neoplastic conditions remain unknown. Alkaline phosphatase is present in human serum in several isoforms. Identification of the different isoforms in serum is complicated by the variety of post-translational modifications. The two major circulating AP isoenzymes, bone and liver, are difficult to distinguish because they are the products of a single gene and differ only by glycosylation. Total serum AP is frequently requested in routine clinical analyses, to determine skeletal and hepatobiliary status. It has been suggested that the various isoforms contributing to the total AP activity provide useful clinical information. Indeed quantitative measurement of bone AP (BAP) activity in serum can provide an index for the rate of bone formation.

Alpha-fetoprotein (AFP) is a major protein of mammalian fetal development and is synthesized mainly by fetal liver and yolk sac. Since hepatoma and yolk sac tumors often produce this protein, it has routinely been used as a tumor marker for diagnosis. In particular AFP is widely used as a serological marker in the diagnosis of hepatocellular carcinoma (HCC) and non-seminomatous germ cell tumours (NSGCT). AFP is also elevated in normal pregnancy, benign liver disease as well as cancer. AFP appears in several disease-associated isoforms that differ in carbohydrate structures. Existing assays cannot easily differentiate between these isoforms.

Other glycoproteins of interest for the present invention include: alpha-1-acid glycoprotein, alpha-1-antitrypsin, haptoglobin, thyroglobulin, prostate specific antigen, HEMPAS erythrocyte band 3 (this is associated with congenital dyserythropoietic anemia type II), PC-1 plasma-cell membrane glycoprotein, CD41 glycoprotein IIb,. CD42b glycocalicin, CD43 leukocyte sialoglycoprotein, CD63 lysosomal-membrane-associated glycoprotein 3, CD66a biliary glycoprotein, CD66f pregnancy specific bi glycoprotein, CD164 multi-glycosylated core protein 24, and the CD235 glycophorin family.

We have now found that the problem of using antibodies or other ligands to discriminate between differently glycosylated protein isoforms in assays may be addressed by the additional use in such assays of a proteolytic enzyme that is capable of breaking down the protein of interest into a plurality of peptide fragments such that the profile of the resulting fragments is characteristic of the glycosylation profile of the analyte in the sample. Thus proteolysis of one isoform of the analyte protein may create fragments which are not produced by proteolysis of the other isoform(s) and which accordingly can be recognised by a specific binding partner for the characteristic fragments or proteolysis of one isoform may produce a set of fragments which shows a different distribution pattern (spectrum) on application of a fragment separation technique (e.g. chromatography, mass spectrometry, etc) to that produced by the fragment set created from the other isoform(s). Particularly suitably, the proteolytic enzyme is one which acts to break the peptide chain at specific sites, e.g. at a specific amino acid residue or a specific amino acid residue sequence. In this way cleavage will occur at such sites when they are exposed in the one isoform of interest but not when they are masked, e.g. by carbohydrate side chains or by different tertiary structure, due to different glycosylation patterns, in other isoforms.

Where the proteolysis of one isoform produces fragments not produced by proteolysis of the other isoform(s), this provides characteristic epitopes and specific binding partners for these characteristic fragments may be used to determine the concentration of the precursor isoform in the sample. Where proteolysis produces similar fragments (e.g. similar in terms of antigenicity or position along a separation axis) but at different relative concentrations, the relative concentrations of two or more such fragments may be determined and used to determine the relative abundance and hence concentrations of the different isoforms in the sample.

Thus viewed from one aspect the invention provides a method for assaying for a protein having at least two isoforms having different glycosylation patterns, said method comprising contacting a sample containing said protein with a proteolytic enzyme, preferably a protein-site specific proteolytic enzyme, and detecting the content or relative content of at least one peptide fragment produced by proteolysis of said protein.

The method of the invention preferably involves determination of an indication of the concentration or relative concentration in the sample or the material from which the sample is derived (e.g. blood) of one isoform of the protein of interest, e.g. a quantitative, semi-quantitative or qualitative indication. Thus for example the concentration of the isoform may be determined, the fraction of the protein existing in that isoform may be determined or the concentration or fraction may be determined simply as being above or below a predetermined threshold value, e.g. a threshold indicative of a healthy or unhealthy state in the patient. Generally however it will be preferred to represent carbohydrate deficiency as the percent (eg mole percent) of the isoforms present that are carbohydrate deficient. To this end the assay method of the invention preferably involves a determination of total content of the glycoprotein, eg by a parallel performance of an assay without the use of a proteolytic enzyme.

Although not strictly necessary when the proteolysis generates fragments characteristic of a particular isoform of the protein of interest, as in many circumstances the sample may contain proteins other than the protein of interest, “noise”, i.e. peptide fragments from such other proteins, may desirably be avoided by separating the protein of interest from the other proteins before contact with the proteolytic enzyme. This may be achieved by chromatography, by selective adsorption onto and release from a substrate, by centrifugation and other standard protein separation techniques. However for ease of assay performance it is preferably achieved by contacting the sample with a substrate to which is bound a specific binding partner for at least the isoforms of interest of the protein of interest and especially preferably a specific binding partner which serves to capture all isoforms of the protein of interest. In this instance the specific binding partner will preferably be an antibody or antibody fragment. The substrate bound protein may then be separated from unbound protein, e.g. by rinsing, and optionally may be released from the substrate before being contacted with the proteolytic enzyme.

Thus viewed from a further aspect the invention provides a kit for an assay method according to the invention, said kit comprising a proteolytic enzyme and a substrate bound specific binding partner (sbp) for at least two and preferably all of the isoforms of the said protein. This substrate bound sbp is preferably one which binds the protein at a site remote from the glycosylation sites. In an especially preferred embodiment, this substrate bound sbp is immobilized on a porous membrane.

Once proteolysis has occurred, the characteristic fragments or characteristic fragmentation pattern may be detected by any conventional technique. However for ease of assay performance detection is preferably of a characteristic fragment using a specific binding partner therefor with the fragment:sbp conjugate then being determined directly or indirectly. Thus in a preferred embodiment the kit of the invention further contains at least one optionally labelled specific binding partner for a peptide fragment producible by the proteolytic action of the enzyme on one isoform of the protein of interest.

The kit also preferably contains instructions for the performance of the assay method and may optionally contain further, optionally labelled, secondary ligands capable of binding to the protein:sbp conjugate and/or the fragment-binding sbp.

Sbp's used in the assay of the invention typically will be antibodies or antibody fragments, oligopeptides, oligonucleotides or small organic molecules. Antibodies and antibody fragments are preferred, especially monoclonal antibodies. In one particular embodiment, antibodies may be raised against immunogenic conjugates of oligopeptides having sequences corresponding to (or similar to) the whole or part of the amino acid sequence of the characteristic protein fragment, e.g. as described in U.S. Pat. No. 5,773,572.

The detection of the conjugates formed by the protein fragments may, as stated above, be direct or indirect. Thus a property (e.g. radiation absorption, emission, or scattering) of a conjugate or of the sbp may be detected, or a further binding reagent with a detectable property or the ability to provoke a detectable property or event may be used. This further binding reagent would be one which binds to such conjugates or to the free sbp or which competes with such conjugates in binding to a further substrate. Such direct and indirect detection of analytes by the use of optionally labelled binding reagents is conventional in the field of diagnostic assays.

The manner in which detection of the protein fragments is made will of course be dependent on the nature of the binding reagents, i.e. whether they are labelled with a reporter moiety such as a radiolabel, a chromophore or a fluorescent dye (i.e. a fluorophore), whether they are enzymatically active (i.e. capable of catalysing a reaction the progress whereof is detectable, e.g. by generation of light or a detectable species), whether they form aggregates which can be detected by light scattering, etc. Such detection systems are conventional in the field of diagnostic assays.

In a preferred embodiment of the method of the invention an sbp for a characteristic protein fragment is immobilized on a porous substrate, e.g. a membrane optionally with a sbp for the protein of interest also immobilized on the same substrate, and following proteolysis and binding to the substrate of the characteristic fragment, a labelled binding partner for the fragment-sbp or the fragment-sbp:fragment conjugate is contacted with the substrate. Following rinsing of the substrate, the substrate-retained label may be read to give a direct or indirect indication of the concentration of the characteristic fragment and hence of the isoform from which it derives.

In an alternative preferred embodiment of the method of the invention a labelled sbp for a characteristic fragment, the conjugate whereof with the fragment is of a size sufficient to be retained by a porous membrane, is contacted with the sample and after proteolysis the sample is passed through the porous membrane (which again optionally can be a membrane on which an sbp for the protein is immobilized). After rinsing, the membrane may be read to give a direct indication of the fragment:labelled-sbp retained thereon and hence an indication of the concentration of the isoform from which the fragment derives.

In a similar embodiment, a competing antigen (e.g. a particle, for example a latex particle carrying antigens) which binds to the fragment-sbp to produce a membrane-retainable conjugate may be used. In this embodiment either or both of the competing antigen and the fragment-sbp should desirably be labelled and the membrane pore size should be sufficiently large as not to retain the unbound fragment-sbp and, where the fragment-sbp is labelled, as not to retain the fragment-sbp:fragment conjugate. After rinsing, the membrane is read to provide an indication of the concentration of the retained antigen:fragment-sbp conjugate and hence indirectly of the fragment.

In these latter two embodiments, the label is preferably a chromophore, a fluorescent dye or, especially, a particulate, e.g. colloidal gold as described in U.S. Pat. No. 5,691,207, U.S. Pat. No. 5,650,333 and EP-A-564449.

These three embodiments are especially suited for use with the assay platform described in WO 02/090995.

As mentioned earlier, the fragments may alternatively be detected by methods which do not require the use of specific binding partners, e.g. by chromatography, mass spectrometry, nmr, etc.

The proteolytic enzyme used in the assay method of the invention may be any enzyme capable of cleaving proteins. Particularly preferably however it is an enzyme capable of cleaving proteins only at specific sites, e.g. adjacent a specific amino acid residue or sequence. One example of such specific proteases is the group of asparaginyl endopeptidases, e.g. legumain, which cleave the amide bonds on the C-terminal side of asparagine moieties. The preparation of such endopeptidases is described for example in U.S. Pat. No. 5,094,952 and they are available commercially from Takara Shuzo Co. Ltd., Kyoto, Japan. Other proteases which may be used include for example achromopeptidase, acylaminopeptidase, aspergillopepsin, carboxypeptidase (A, B or C), cathepsin (B, D, G or H), chymopapain, dipeptidyl-peptidase (I and IV), endopeptidase K, endoproteinase Arg-C, enteropeptidase, ficain, gelatinase, γ-Glu-X carboxypeptidase, glutamyl endopeptidase, leucyl aminopeptidase, membrane alanyl aminopeptidase, membrane Pro-C carboxypeptidase, microbial collagenase, multicatalytic endopeptidase complex, pancreatic elastase, pepsin A, peptidyl-Asp metalloendopeptidase, peptidyl-dipeptidase, plasma kallikrein, plasmin, t-plasminogen activator, u-plasminogen activator, pyroglutamyl-peptidase, renin, retropepsin, stem bromelain, subtilisin, thermolysin, thrombin, tissue kallikrein, chymotrypsin, calpain, proteinase K, clostripain, coagulation factor Xa, trypsin, and papain. If desired two or more such proteases can be used simultaneously or sequentially. The use of chymotrypsin is especially preferred.

In the method of the invention, the protein is preferably incubated with the proteolytic enzyme for a period and under conditions such that the protein is cleaved so releasing fragments characteristic of the different isoforms from which they derive.

Typically, incubation will be for 1 to 120 minutes, preferably 5 to 40 minutes, especially preferably at a temperature of from ambient to 42° C., particularly ambient to 38° C.

With any particular protein of interest, in order to decide which fragments to use as analytes, it will generally be desirable to cleave the glycosylated and non-glycosylated isoforms, and compare the fragments produced using chromatography to separate them. Spectroscopy can then be used to identify the appropriate fragment to choose and, if the protein sequence is known and the protease is site-specific in its cleavage, the chosen fragment sequence can be identified from the set of possible fragments. With the fragment thus identified, sbp's for it may then be generated using conventional techniques.

Especially preferably, the characteristic fragments are identified using a dual modality separation and spectroscopic technique, e.g. combined chromatography and mass spectrometry or nmr.

In one embodiment of the invention, detection may be effected using surface plasmon resonance (SPR), a non-invasive optical technique in which the SPR response reflects the change in mass concentration at the detector surface as molecules bind or dissociate.

SPR may be carried out using the proprietary system known as Biacore analysis (available from Biacore AB, Uppsala, Sweden).

The method of the invention is particularly suited for use in assaying multiple samples, eg using a multiwell microtitre plate format (typically an n×m well plate where n and m are positive integers having values up to 20, especially a 96-well microtitre plate).

The samples used in the assay method of the invention will typically be samples of or derived from a body tissue, organ or fluid (eg urine, saliva, mucous, blood, etc). Preferably the sample is blood or derived from blood, eg serum. The species of the subject from which the sample is taken is preferably a mammalian, reptilian, avian or fish or shellfish species, more preferably mammalian (especially human).

Where the glycoprotein is cell bound or cell-encapsulated the sample may be treated in conventional fashion to release the glycoprotein. Similarly the glycoprotein may if desired be metallated (eg by addition of iron ions where the protein is an iron-binding protein), demetallated or denatured. The precise nature in which the sample is pretreated will thus depend on the particular glycoprotein being assayed for.

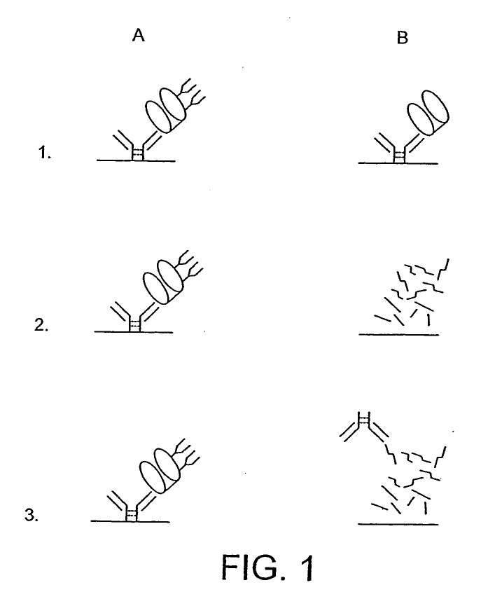

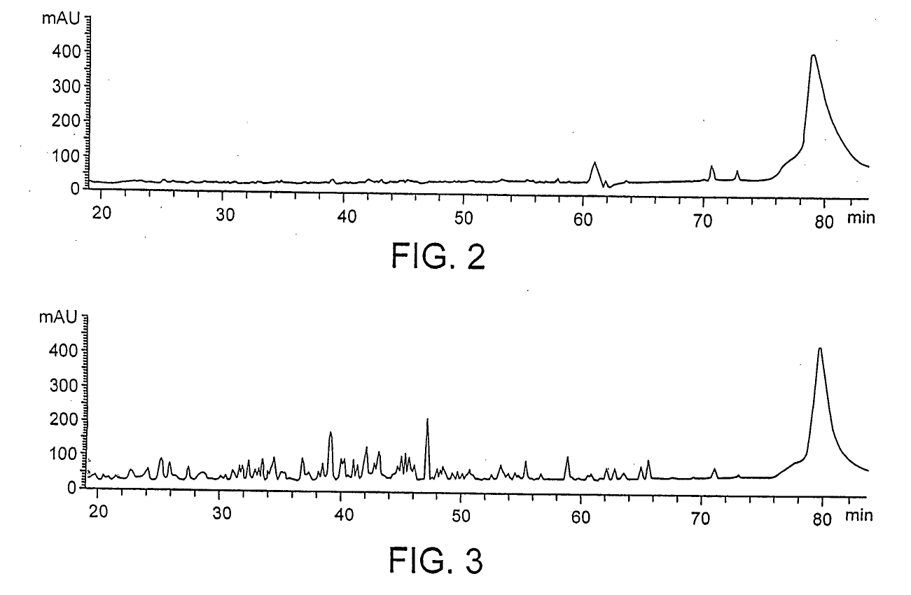

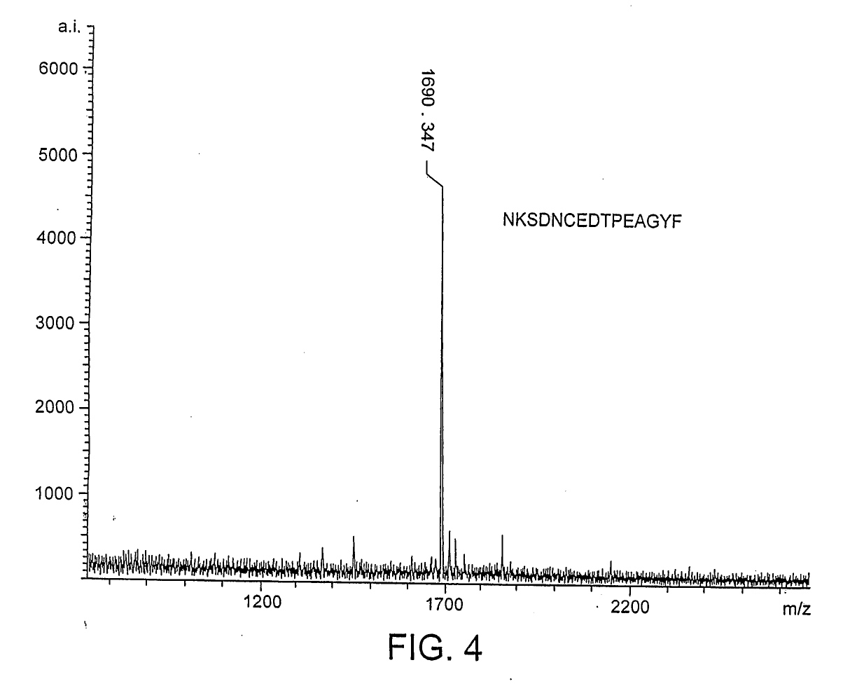

An example of an assay according to the invention for transferrin is illustrated schematically in FIG. 1 of the accompanying drawings. FIGS. 2 and 3 of the accompanying drawings show reversed phase HPLC plots of the protein fragments obtained by digesting glycosylated and non-glycosylated transferrin with chymotrypsin. FIG. 1 shows the principle of the assay according to the invention i.e. how asialotransferrin can be distinguished from normal transferrin by the differences in their proteolysis. Column A refers to tetrasialotransferrin and column B shows asialotransferrin. Step 1 illustrates the solid-phase capture of transferrin from serum. Both non-glycosylated transferrin isoforms are captured. Step 2 is digestion of the antibody-transferrin complex. Only non-glycosylated is digested to yield the unique fragmentation profile and Step 3 is detection of specific peptide fragment. The antibody will recognize the epitope on the peptide, but not within the intact fully-glycosylated transferrin. FIG. 2 shows the plot for glycosylated transferrin and FIG. 3 shows the plot for non-glycosylated transferrin. As can be seen, there are several fragments characteristic of (i.e. essentially unique to) the non-glycosylated isoform. FIG. 4 is an example of a peptide sequence for non-glycosylated transferrin produced by chymotrypsin cleavage and identified by MALDI-TOF and MS-MS.

Tables 1 to 3 below sets out the peptide fragments of molecular weight above 500 g/mol which theoretically could be achieved by cleavage of transferrin with chymotrypsin, trypsin and Lys-C respectively. Antibodies to such fragments can readily be produced by antibody generation using an immunogenic conjugate of the fragment to a carrier molecule, e.g. as described in U.S. Pat. No. 5,773,572. The assay method however could simply be effected using HPLC and determining the extent to which peaks characteristic of the non-glycosylated transferrin are present.

TABLE 1

| mass | sequence position | peptide sequence |
|---|---|---|
| 2527.2 | 359-382 | SVNSVGKIECVSAETTEDCIAKIM |
| 2456.4 | 580-601 | ARAPNHAVVTRKDKEACVHKIL |
| 2045.9 | 327-344 | REGTCPEAPTDECKPVKW |
| 2003.0 | 533-550 | VKHQTVPQNTGGKNPDPW |
| 1936.0 | 27-45 | KSVIPSDGPSVACVKKASY |
| 1686.9 | 445-460 | KGKKSCHTAVGRTAGW |
| 1645.8 | 47-62 | DCIRAIAANEADAVTL |
| 1568.6 | 155-170 | SGSCAPCADGTDFPQL |
| 1542.6 | 413-426 | NKSDNCEDTPEAGY |
| 1539.7 | 9-22 | CAVSEHEATKCQSF |
| 1382.7 | 244-256 | AQVPSHTVVARSM |
| 1365.6 | 481-494 | SEGCAPGSKKDSSL |
| 1236.6 | 229-238 | DNTRKPVDEY |
| 1193.6 | 565-574 | DGTRKPVEEY |
| 1187.7 | 428-439 | AVAVVKKSASDL |
| 1167.6 | 137-146 | CDLPEPRKPL |
| 1120.6 | 97-107 | AVAVVKKDSGF |
| 1100.4 | 609-619 | GSNVTDCSGNF |
| 1086.6 | 113-122 | RGKKSCHTGL |
| 1080.5 | 215-223 | ANKADRDQY |
| 1053.4 | 506-514 | CEPNNKEGY |
| 1031.5 | 469-476 | NKINHCRF |
| 1008.5 | 86-94 | GSKEDPQTF |
| 1000.6 | 1-8 | VPDKTVRW |
| 938.5 | 275-282 | GKDKSKEF |
| 881.5 | 663-670 | RKCSTSSL |
| 879.4 | 196-204 | KDGAGDVAF |
| 873.4 | 268-274 | NQAQEHF |
| 864.4 | 525-532 | VEKGDVAF |
| 848.4 | 623-629 | RSETKDL |
| 840.4 | 286-293 | SSPHGKDL |
| 840.3 | 174-182 | CPGCGCSTL |
| 831.5 | 205-211 | VKHSTIF |
| 821.4 | 632-638 | RDDTVCL |
| 809.4 | 602-607 | RQQQHL |
| 805.4 | 642-647 | HDRNTY |
| 789.5 | 78-84 | KPVVAEF |
| 786.5 | 320-326 | VTAIRNL |
| 778.4 | 147-153 | EKAVANF |
| 778.4 | 348-353 | SHHERL |
| 761.4 | 296-302 | KDSAHGF |
| 727.4 | 304-309 | KVPPRM |
| 707.3 | 383-389 | NGEADAM |
| 700.4 | 656-662 | VKAVGNL |
| 680.3 | 354-358 | KCDEW |
| 668.3 | 555-559 | NEKDY |
| 661.4 | 398-404 | IAGKCGL |
| 633.3 | 123-128 | GRSAGW |
| 626.4 | 129-134 | NIPIGL |
| 618.3 | 257-262 | GGKEDL |
| 615.3 | 239-243 | KDCHL |
| 570.2 | 672-676 | EACTF |
| 558.2 | 23-26 | RDHM |
| 557.3 | 575-579 | ANCHL |
| 528.3 | 73-77 | APNNL |

TABLE 2

| mass | sequence position | peptide sequence |
|---|---|---|
| 4646.0 | 149-193 | AVANFFSGSCAPCADGTDFPQLCQLCPGCGCSTLNQYFGYSGAFK |
| 3954.0 | 51-88 | AIAANEADAVTLDAGLVYDAYLAPNNLKPVVAEFYGSK |
| 2401.1 | 603-623 | QQQHLFGSNVTDCSGNFCLFR |
| 2159.0 | 381-401 | IMNGEADAMSLDGGFVYIAGK |
| 2114.1 | 125-143 | SAGWNIPIGLLYCDLPEPR |
| 2070.0 | 260-276 | EDLIWELLNQAQEHPGK |
| 2014.9 | 415-433 | SDNCEDTPEAGYFAVAVVK |
| 1703.8 | 328-343 | EGTCPEAPTDECKPVK |
| 1632.8 | 240-254 | DCHLAQVPSHTVVAR |
| 1629.8 | 89-102 | EDPQTFYYAVAVVK |
| 1611.7 | 366-380 | ECVSAETTEDCIAK |
| 1592.7 | 497-511 | LCMGSGLNLCEPNNK |
| 1577.8 | 457-470 | TAGWNIPMGLLYNK |
| 1529.8 | 569-581 | KPVEEYANCHLAR |
| 1520.6 | 476-489 | FDEPPSEGCAPGSK |
| 1482.7 | 221-232 | DQYELLCLDNTR |
| 1478.7 | 313-324 | MYLGYEYVTAIR |
| 1419.7 | 402-414 | CGLVPVLAENYNK |
| 1417.6 | 665-677 | CSTSSLLEACTPR |
| 1358.7 | 28-41 | SVIPSDGPSVACVK |
| 1297.6 | 558-568 | DYELLCLDGTR |
| 1283.6 | 512-522 | EGYYGYTGAPR |
| 1276.6 | 281-291 | EPQLFSSPHGK |
| 1273.7 | 207-217 | HSTIFENLANK |
| 1260.6 | 8-18 | WCAVSEHEATK |
| 1249.6 | 435-445 | SASDLTWDNLK |
| 1223.5 | 355-365 | CDEWSVNSVGK |
| 1195.6 | 104-113 | DSGFQMNQLR |
| 1166.6 | 535-545 | HQTVPQNTGGK |
| 1138.5 | 344-352 | WCALSHHER |
| 1000.5 | 650-657 | YLGEEYVK |
| 978.5 | 197-206 | DGAGDVAFVK |
| 964.5 | 582-590 | APNHAVVTR |
| 940.5 | 43-50 | ASYLDCIR |
| 878.5 | 233-239 | KPVDEYK |
| 874.4 | 297-304 | DSAHGFLK |
| 864.4 | 633-640 | DDTVCLAK |
| 830.4 | 117-124 | SCHTGLGR |
| 830.4 | 449-456 | SCHTAVGR |
| 827.4 | 546-552 | NPDPWAK |
| 735.4 | 528-534 | GDVAFVK |
| 686.3 | 594-599 | EACVHK |
| 663.4 | 628-632 | DLLFR |
| 654.3 | 645-649 | NTYEK |
| 652.3 | 491-496 | DSSLCK |
| 642.3 | 471-475 | INHCR |
| 640.3 | 19-23 | CQSFR |
| 635.4 | 292-296 | DLLFK |
| 629.4 | 658-663 | AVGNLR |
| 617.3 | 553-557 | NLNEK |
| 614.4 | 144-148 | KPLEK |
| 591.3 | 523-527 | CLVEK |
| 540.3 | 641-644 | LHDR |
| 530.2 | 24-27 | DHMK |

TABLE 3

| mass | sequence position | peptide sequence |
|---|---|---|
| 4875.5 | 43-88 | ASYLDCIRAIAANEADAVTLDAGLVYDAYLAPNNLKPVVAEFYGSK |
| 4646.0 | 149-193 | AVANFFSGSCAPCADGTDFPQLCQLCPGCGCSTLNQYFGYSGAFK |
| 3881.9 | 558-591 | DYELLCLDGTRKPVEEYANCHLARAPNHAVVTRK |
| 3546.7 | 313-343 | MYLGYEYVTAIRNLREGTCPEAPTDECKPVK |
| 3520.8 | 117-148 | SCHTGLGRSAGWNIPIGLLYCDLPEPRKPLEK |
| 3228.6 | 600-627 | ILRQQQHLFGSNVTDCSGNPCLPRSETK |
| 2684.3 | 218-239 | ADRDQYELLCLDNTRKPVDEYK |
| 2389.2 | 449-470 | SCHTAVGRTAGWNIPMGLLYNK |
| 2159.0 | 381-401 | IMNGEADAMSLDGGFVYIAGK |
| 2143.9 | 471-489 | INHCRFDEFFSEGCAPGSK |
| 2093.0 | 240-259 | DCHLAQVPSHTVVARSMGGK |
| 2070.0 | 260-276 | EDLIWELLNQAQEHFGK |
| 2014.9 | 415-433 | SDNCEDTPEAGYFAVAVVK |
| 1855.9 | 512-527 | EGYYGYTGAFRCLVEK |
| 1670.8 | 665-679 | CSTSLLEACTFRRP |
| 1629.8 | 89-102 | EDPQTFYYAVAVVK |
| 1616.8 | 5-18 | TVRWCAVSEHEATK |
| 1611.7 | 366-380 | IECVSAETTEDCLAK |
| 1592.7 | 497-511 | LCMGSGLNLCEPNNK |
| 1508.8 | 628-640 | DLLFRDDTVCLAK |
| 1419.7 | 402-414 | CGLVPVLAENYNK |
| 1380.7 | 104-115 | DSGFQMNQLRGK |
| 1379.7 | 344-354 | WCALSHHERLK |
| 1358.7 | 28-41 | SVIPSDGPSVACVK |
| 1276.6 | 281-291 | EFQLFSSPHGK |
| 1273.7 | 207-217 | HSTIFENLANK |
| 1249.6 | 435-445 | SASDLTWDNLK |
| 1223.5 | 355-365 | CDEWSVNSVGK |
| 1175.6 | 641-649 | LHDRNTYEK |
| 1166.6 | 535-545 | HQTVPQNTGGK |
| 1151.5 | 19-27 | CQSFRDHMK |
| 1000.5 | 650-657 | YLGEEYVK |
| 978.5 | 197-206 | DGAGDVAFVK |
| 913.5 | 305-312 | VPPRMDAK |
| 874.4 | 297-304 | DSAHGFLK |
| 827.4 | 546-552 | NPDPWAK |
| 757.5 | 658-664 | AVGNLRK |
| 735.4 | 528-534 | GDVAFVK |
| 686.3 | 594-599 | EACVHK |
| 652.3 | 491-496 | DSSLCK |
| 636.4 | 292-296 | DLLFK |
| 617.3 | 553-557 | NLNEK |

FIG. 4 shows an example of a peptide sequence determined experimentally from MALDI-TOF and electrospray MS-MS that is specifically release from the enzymatic cleavage of non-glycosylated transferrin by chymotrypsin. This sequence is NKSDNCEDTPEAGYF

This sequence represents an ideal candidate for raising monoclonal antibodies that would only recognise the cleavage products of non-glycosylated transferrin. This sequence compounds to a deamidated non-glycosylated 15 residue peptide with a monoisotopic mass value of 1690 determined from MALDI-TOF MS of a peak fraction isolated from reverse phase hplc. The peptide fragment compounds closely to the ninth fragment listed in Table 1.

### EXAMPLE 1

Assay

1. Add 150 μL of serum test sample or control to individual wells of a 96 well microtitre plate previously coated with anti-transferrin monoclonal antibody, and incubate with gentle mixing at 37° C. for 30 minutes.
2. Wash the wells twice with 200 μL of 100 mM Tris HCl buffer pH 7.8 containing 0.05% Tween 20, followed by 1 wash with 200 μL of 100 mM Tris HCl buffer pH 7.8 without Tween 20.
3. Add 150 μL of prewarmed 100 mM Tris HCl buffer pH 7.8 to each well followed by 10 μL sequencing grade chymotrypsin (2 μg/μL), and incubate at 37° C. for 30 minutes.
4. Stop the reaction by adding 10 μL of pre-cooled acetic acid at 4° C.
5. Add 30 μL 100 mM TCEP and incubate for a further 10 minutes.
6. Transfer the contents of each well to a new well precoated with peptide.
7. Add 50 μL of ¹²⁵I labelled anti-peptide antibody and incubate at 37° C. for 60 minutes.
8. Wash wells 3 times and 100 mM Tris HCl buffer pH 7.8 containing 0.05% Tween 20 and determine the amount of ¹²⁵I labelled anti-peptide antibody bound to the plate.
9. Amount of bound antibody is compared with a standard curve to determine the amount of peptide and hence CDT in the original sample.

### EXAMPLE 2

Fluorescence Polarised Immunoassay

1. Add 50 μL of serum test sample or control to an appropriate receptacle and incubate with 150 μL of prewarmed 100 mM Tris HCl buffer pH 7.8 containing sequencing grade chymotrypsin (40 μg) at 35° C. for 30 minutes.
2. Stop the reaction by adding a standard anti-chymotrypsin inhibitor (e.g. 100 μM TPCK or Aprotonin).
3. Add 30 μL 100 mM TCEP and incubate for a further 10 minutes.
4. Transfer the proteolytic cleavage mixture to a new well containing a pre-defined amount of fluorescent labelled peptide.
5. Measure the degree of polarized fluorescence, mP.
6. Add 50 μL of anti-peptide antibody and incubate at 35° C. for 5 minutes.
7. Make a second measurement of polarized fluorescence, mP′.
8. The difference in polarized fluorescence, which reflects the relative amount of peptide binding to the antibody, is compared with a standard curve to determine the amount of peptide and hence CDT in the original sample.

1. A method for assaying for a protein having at least two isoforms having different glycosylation patterns, said method comprising: contacting a sample containing said protein with a protein site specific proteolytic enzyme, and detecting the content or relative content of at least one peptide fragment which is produced by proteolysis of one isoform but not by proteolysis of the other isoform(s), through the use of a specific binding partner (sbp) for that fragment or fragments.

2. A method as claimed in claim 1 wherein said protein is selected from transferrin, alkaline phosphatase, chorionic gonadotropin and alpha-fetoprotein.

3. A method as claimed claim 1 wherein the sbp is an antibody or antibody fragment.

4. A method as claimed in claim 1 wherein the protein of interest is separated from other proteins before contact with the proteolytic enzyme.

5. A kit for an assay method according to claim 1, said kit comprising a proteolytic enzyme and a substrate bound specific binding partner (sbp) for at least two of the isoforms of the said protein.

6. A kit as claimed in claim 5 wherein said sbp is for all of the isoforms of said protein.

7. A kit as claimed in claim 5 wherein said sbp is an antibody or antibody fragment.

8. A kit as claimed in claim 5 wherein said sbp binds the protein at a site remote from the glycosylation sites.

9. A kit as claimed in claim 5 wherein detection is of a characteristic peptide fragment: sbp conjugate.

10. A kit as claimed in claim 5 wherein said sbp is labelled.

11. A kit as claimed in claim 5 wherein said sbp is labelled with a fluorescent dye.
